(19) United States
(12) Patent Application Publication
(10) Pub. No.: US 2005/0287575 A1
(43) Pub. Date: Dec. 29, 2005

(54) System and method for improved genotype calls using microarrays

(75) Inventors: Xiaojun Di, Sunnyvale, CA (US); Simon Cawley, Oakland, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/157,768

(22) Filed: Jun. 21, 2005

Related U.S. Application Data
(63) Continuation-in-part of application No. 10/986,963, filed on Nov. 12, 2004.
Application No. 10/986,963 is a continuation-in-part of application No. 10/657,481, filed on Sep. 8, 2003.
(60) Provisional application No. 60/581,773, filed on Jun. 22, 2004.

(51) Int. Cl. C12Q 1/68; G06F 19/00

(52) U.S. Cl. 435/6; 702/20

(57) ABSTRACT

An embodiment of a method for calling the genotype of a biological sequence is described that comprises receiving sets of intensity data each comprising an intensity value for each probe feature associated with a probe set disposed on a probe array; independently applying filters to the intensity values of a probe set associated with a forward strand and of a probe set associated with a reverse strand, where the probe sets interrogate the same sequence position; independently applying models to the filtered intensity values for the forward strand and the reverse strand, where the models produce a genotype call for each strand; combining the genotype call for the forward strand and the genotype call for the reverse strand to generate a final genotype call; and testing the reliability of the final genotype call.

### RELATED APPLICATIONS

The present application claims priority to U.S. Provisional Patent Application Ser. No. 60/581,773, titled “System and Method for Improved Genotype Calls Using Microarrays, filed Jun. 22, 2004; which is hereby incorporated by reference herein in its entirety for all purposes. The present application is also a continuation-in-part of U.S. patent application Ser. No. 10/986,963, titled “System, Method, and Computer Software Product for Generating Genotype Calls”, filed Nov. 12, 2004; which is a continuation-in-part of U.S. patent application Ser. No. 10/657,481, titled “System, Method, and Computer Software Product for Analysis and Display of Genotyping, Annotation, and Related Information”, filed Sep. 8, 2003, each of which is also hereby incorporated by reference herein in its entirety for all purposes.

### BACKGROUND

1. Field of the Invention

The present invention relates to the field of bioinformatics. In particular, the present invention relates to systems, and methods for generating improved genotype calls determined from the analysis of an organism's genotype using biological probe arrays.

2. Related Art

Synthesized nucleic acid probe arrays, such as Affymetrix® GeneChip® probe arrays, and spotted probe arrays, have been used to generate unprecedented amounts of information about biological systems. For example, the GeneChip® Human Genome U133 Plus 2.0 probe array available from Affymetrix, Inc. of Santa Clara, Calif., is comprised of a single microarray containing over 1,000,000 unique oligonucleotide features covering more than 47,000 transcripts that represent more than 33,000 human genes. Analysis of expression data from such microarrays may lead to the development of new drugs and new diagnostic tools.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

An embodiment of a method for calling the genotype of a biological sequence is described that comprises receiving sets of intensity data each comprising an intensity value for each probe feature associated with a probe set disposed on a probe array; independently applying filters to the intensity values of a probe set associated with a forward strand and of a probe set associated with a reverse strand, where the probe sets interrogate the same sequence position; independently applying models to the filtered intensity values for the forward strand and the reverse strand, where the models produce a genotype call for each strand; combining the genotype call for the forward strand and the genotype call for the reverse strand to generate a final genotype call; and testing the reliability of the final genotype call.

Also, an embodiment of a system for calling the genotype of a biological sequence is described that comprises a data manager that receives one or more sets of intensity data each comprising an intensity value for each of a plurality of probe features, wherein each probe feature is associated with a probe set disposed on a probe array; data filters that independently apply one or more filters to the intensity values of the probe sets associated with a forward strand and a reverse strand, where the probe sets for the forward and reverse strands interrogate a same sequence position; a comparator that applies models to the filtered intensity values for each of the forward strand and the reverse strand, where the models produce a genotype call for both the forward and reverse strands independently, and further that the comparator combines the genotype call for the forward strand and the genotype call for the reverse strand to generate a final genotype call for the same sequence position; and a reliability tester that tests the reliability of the final genotype call.

In addition, an embodiment of a method for calling the genotype of a biological sequence is described that comprises receiving a one or more sets of intensity data each comprising an intensity value for each of a plurality of probe features, where each probe feature is associated with a probe set disposed on a probe array; applying one or more models to each of the intensity values for the probe set associated with each of a forward strand and a reverse strand, where the models produce a genotype call for the forward strand and a genotype call for the reverse strand; and combining the genotype call for the forward strand and the genotype call for the reverse strand to generate a final genotype call for the same sequence position.

Further, an embodiment of a system for calling the genotype of a biological sequence is described that comprises a computer comprising system memory having executable code stored thereon, where the executable code performs a method, comprising; receiving a one or more sets of intensity data each comprising an intensity value for each of a plurality of probe features, where each probe feature is associated with a probe set disposed on a probe array; independently applying one or more filters to the intensity values of a probe set associated with a forward strand and the intensity values of a probe set associated with a reverse strand, where the probe sets for the forward and reverse strands interrogate a same sequence position; independently applying one or more models to the filtered intensity values for each of the forward strand and the reverse strand, where the models produce a genotype call for the forward strand and a genotype call for the reverse strand; combining the genotype call for the forward strand and the genotype call for the reverse strand to generate a final genotype call for the same sequence position; and testing the reliability of the final genotype call.

The above implementations are not necessarily inclusive or exclusive of each other and may be combined in any manner that is non-conflicting and otherwise possible, whether they are presented in association with a same, or a different, aspect of implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

In the drawings, like reference numerals indicate like structures or method steps and the leftmost digit of a reference numeral indicates the number of the figure in which the referenced element first appears (for example, the element 120 appears first in FIG. 1). In functional block diagrams, rectangles generally indicate functional elements, parallelograms generally indicate data, and rectangles with a pair of double borders generally indicate predefined functional elements. These conventions, however, are intended to be typical or illustrative, rather than limiting.

FIG. 1 is a functional block diagram of one embodiment of a computer system including illustrative embodiments of instrument control and image processing executables and display/output devices including graphical user interfaces;

FIG. 2 is a functional block diagram of one embodiment of the computer system of FIG. 1 connected to a user-side Internet client and database server via a network for communication over the Internet;

FIG. 3 is a functional block diagram of one embodiment of the instrument control and image processing executables of FIG. 1 including illustrative embodiments of a sequence data manager and an output manager;

FIG. 4 is a functional block diagram of one embodiment of a method for making genotype calls; and

FIG. 5 is a simplified graphical representation of one embodiment of a graphical user interface for presenting genotype calls to a user.

### DETAILED DESCRIPTION

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841; WO 00/58516; U.S. Pat. Nos. 5,143,854; 5,242,974; 5,252,743; 5,324,633; 5,384,261; 5,405,783; 5,424,186; 5,451,683; 5,482,867; 5,491,074; 5,527,681; 5,550,215; 5,571,639; 5,578,832; 5,593,839; 5,599,695; 5,624,711; 5,631,734; 5,795,716; 5,831,070; 5,837,832; 5,856,101; 5,858,659; 5,936,324; 5,968,740; 5,974,164; 5,981,185; 5,981,956; 6,025,601; 6,033,860; 6,040,193; 6,090,555; 6,136,269; 6,269,846; and 6,428,752; in PCT Applications Nos. PCT/US99/00730 (International Publication No. WO 99/36760); and PCT/US01/04285 (International Publication No. WO 01/58593); which are all incorporated herein by reference in their entirety for all purposes.

Patents that describe synthesis techniques in specific embodiments include U.S. Pat. Nos. 5,412,087; 6,147,205; 6,262,216; 6,310,189; 5,889,165; and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992; 6,013,449; 6,020,135; 6,033,860; 6,040,138; 6,177,248; and 6,309,822. Genotyping and uses therefore are shown in U.S. Ser. Nos. 10/442,021; 10/013,598 (U.S. patent application Publication 20030036069); and U.S. Pat. Nos. 5,856,092; 6,300,063; 5,858,659; 6,284,460; 6,361,947; 6,368,799; and 6,333,179. Other uses are embodied in U.S. Pat. Nos. 5,871,928; 5,902,723; 6,045,996; 5,541,061; and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202; 4,683,195; 4,800,159; 4,965,188; and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909; 5,861,245) and nucleic acid based sequence amplification (NABSA). (See, U.S. Pat. Nos. 5,409,818; 5,554,517; and 6,063,603, each of which is incorporated herein by reference). Other amplification methods that may be used are described in, U.S. Pat. Nos. 5,242,794; 5,494,810; 4,988,617; and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135; 09/920,491 (U.S. patent application Publication 20030096235); Ser. No. 09/910,292 (U.S. patent application Publication 20030082543); and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928; 5,874,219; 6,045,996; 6,386,749; and 6,391,623 each of which are incorporated herein by reference.

The present invention also contemplates signal detection of hybridization between ligands in certain preferred embodiments. For example, methods and apparatus for signal detection and processing of intensity data are disclosed in, U.S. Pat. Nos. 5,143,854; 5,547,839; 5,578,832; 5,631,734; 5,800,992; 5,834,758; 5,856,092; 5,902,723; 5,936,324; 5,981,956; 6,025,601; 6,090,555; 6,141,096; 6,171,793; 6,185,030; 6,201,639; 6,207,960; 6,218,803; 6,225,625; 6,252,236; 6,335,824; 6,403,320; 6,407,858; 6,472,671; 6,490,533; 6,650,411; and 6,643,015, in U.S. patent application Ser. Nos. 10/389,194; 60/493,495; and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,733,729; 5,593,839; 5,795,716; 5,733,729; 5,974,164; 6,066,454; 6,090,555; 6,185,561; 6,188,783; 6,223,127; 6,228,593; 6,229,911; 6,242,180; 6,308,170; 6,361,937; 6,420,108; 6,484,183; 6,505,125; 6,510,391; 6,532,462; 6,546,340; and 6,687,692.

Additionally, the present invention may have preferred embodiments that include methods for providing genetic information over networks such as the Internet as shown in U.S. Ser. Nos. 10/197,621; 10/063,559 (U.S. Publication No. 20020183936); Ser. Nos. 10/065,856; 10/065,868; 10/328,818; 10/328,872; 10/423,403; and 60/482,389.

### b) Definitions

The term “admixture” refers to the phenomenon of gene flow between populations resulting from migration. Admixture can create linkage disequilibrium (LD).

The term “allele’ as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a I column by m row matrix of the building blocks to be added. The switch matrix is all or a subset of the binary numbers, preferably ordered, between I and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids. The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified.

Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na+], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip® Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991); LNAs, as described in Koshkin et al. Tetrahedron 54:3607-3630, 1998, and U.S. Pat. No. 6,268,490; aptamers, and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “mendelian inheritance” as used herein refers to a set of commonly held principles that underlie the theories of genetic inheritance from parent to offspring that include units of inheritance that are passed intact from one generation to the next.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate.

The term “nucleic acids” as used herein may include a polymeric form of nucleotides of any length, any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component (PNAs), other natural, chemically or biochemically modified, non-natural, or derivatized nucleotide bases and any chemical variants thereof, such as methylated, hydroxymethylated or glucosylated forms of these bases, and the like. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) Embodiments of the Present Invention

User Computer 100: User computer 100 may be a computing device specially designed and configured to support and execute some or all of the functions of instrument control and image processing applications 199, described below. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Processor 105 executes operating system 110, which may be, for example, a Windows®-type operating system (such as Windows NT® 4.0 with SP6a, or Windows® XP) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, USB drive, or a diskette drive. Such types of memory storage device 125 typically read from, and/or write to, a program storage medium (not shown) such as, respectively, a compact disk, magnetic tape, removable hard disk, USB drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 175, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

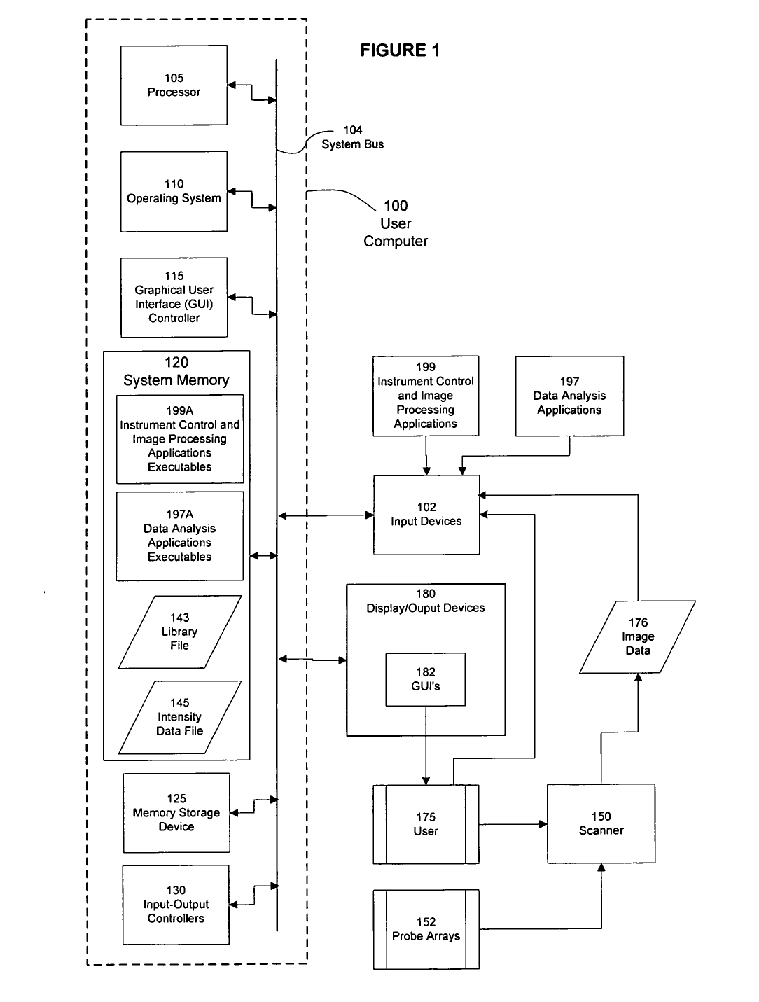

Scanner 150: Labeled targets hybridized to probe arrays may be detected using various devices, sometimes referred to as scanners, as described above with respect to methods and apparatus for signal detection. An illustrative device is shown in FIG. 1 as scanner 150. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

For example, scanner 150 provides a signal representing the intensities (and possibly other characteristics, such as color that may be associated with a detected wavelength) of the detected emissions or reflected wavelengths of light, as well as the locations on the substrate where the emissions or reflected wavelengths were detected. Typically, the signal includes intensity information corresponding to elemental sub-areas of the scanned substrate. The term “elemental” as used herein generally refers to the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. The size of a pixel may vary in different embodiments and could include a 2.5 μm, 1.5 μm, 1.0 μm, or sub-micron pixel size. Two examples where the signal may be incorporated into data are data files in the form *.dat or *.tif as generated respectively by Affymetrix® Microarray Suite (described in U.S. patent application Ser. No. 10/219,882, which is hereby incorporated by reference herein in its entirety for all purposes) or Affymetrix® GeneChip(200 Operating Software (described in U.S. patent application Ser. No. 10/764,663, which is hereby incorporated by reference herein in its entirety for all purposes ) based on images scanned from GeneChip® arrays, and Affymetrix® Jaguar™ software (described in U.S. patent application Ser. Nos. 09/682,071, and 09/682,076; and U.S. Pat. No. 6,789,040; each of which is hereby incorporated by reference herein in its entirety for all purposes) based on images scanned from spotted arrays. Examples of scanner systems that may be implemented with embodiments of the present invention include U.S. patent application Ser. No. 10/389,194, incorporated by reference above. Other examples also include U.S. patent application Ser. Nos. 10/846,261, and 10/913,102; and U.S. Provisional Patent Application Ser. No. 60/623,390, titled “System, Method and Product for Multiple Wavelength Detection Using Single Source Excitation”, filed Oct. 29, 2004; each of which is hereby incorporated by reference by reference herein in its entirety for all purposes.

Probe Arrays 152: An illustrative example of probe array 152 is provided in FIG. 1. Descriptions of probe arrays are provided above with respect to “Nucleic Acid Probe arrays” and other related disclosure. In various implementations, probe array 152 may be disposed in a cartridge or housing such as, for example, the GeneChip® probe array available from Affymetrix, Inc. of Santa Clara, Calif. Further examples of housings for biological probe arrays may be found in U.S. Pat. Nos. 5,945,334; 6,287,850; 6,399,365; 6,551,817; and 6,733,977, each of which is hereby incorporated by reference herein in its entirety for all purposes.

Instrument control and image processing applications 199: Instrument control and image processing applications 199 may be any of a variety of known or future instrument control and image processing applications. Examples of applications 199 include Affymetrix® Microarray Suite, Affymetrix® GeneChip® Operating Software (hereafter referred to as GCOS), and Affymetrix® Jaguar™ software. Applications 199 may be loaded into system memory 120 and/or memory storage device 125 through one of input devices 102.

Embodiments of applications 199 include executable code being stored in system memory 120, illustrated in FIG. 1 as instrument control and analysis applications executables 199A. Applications 199 may provide a modular interface for one or more computers or workstations and one or more servers, as well as one or more instruments. In the presently described implementation, the interface may communicate with and control one or more elements of the one or more servers, one or more workstations, and the one or more instruments.

In some embodiments, image data 176 is acquired from scanner 150 and operated upon by applications 199 to generate intermediate results. Examples of intermediate results include so-called cell intensity files (*.cel) and chip files (*.chp) generated by Affymetrix® GeneChip® Operating Software or Affymetrix® Microarray Suite (as described, for example, in U.S. patent application, Ser. Nos. 10/219,882, and 10/764,663, both of which are hereby incorporated herein by reference in their entireties for all purposes) and spot files (*.spt) generated by Affymetrix® Jaguar™ software (as described, for example, in PCT Application PCT/US 01/26390 and in U.S. patent applications, Ser. Nos. 09/681,819, 09/682,071, 09/682,074, and 09/682,076, all of which are hereby incorporated by reference herein in their entireties for all purposes). For example, intensity data file 145, as illustrated in FIG. 1, may include a cel file processed by executables 199A from image data 176. In the present example, each of files 145 may comprise, for each probe feature scanned by scanner 150, a single value representative of the intensities of pixels measured by scanner 150 for that probe feature. Thus, for instance each value is representative of the presence or absence of tagged or labeled target molecules present in the sample that hybridized to the corresponding probe feature. Many probe molecules complementary to each target molecule may be present in each probe feature, as a probe feature on a GeneChip® probe array may include, for example, millions of oligonucleotides designed to detect the target molecules.

For example, applications 199 may process image data 176 by initially determining the positional location of features in the image. In some embodiments the processing includes placing what is referred to as a grid on the image where each of the features of a properly positioned grid may be bounded by the lines of the grid. Typically, control features comprising high contrast patterns may be disposed upon probe array 152 that hybridize to control targets in a sample and provide a measure of hybridization efficiency as well as positional “anchors” in the resulting image for positional recognition and grid placement. Applications 199 may employ an image deconvolution algorithm to the image in order to determine the location of the control features in the image and their relationship to one another. Thus, applications 199 may determine the proper placement of a grid using the locations of a plurality of control feature. In some embodiments, the control feature may comprise small “checkerboard” type patterns placed at each corner of what may be referred to as the active area (i.e. area where the probe features are disposed). It may also be advantageous in some embodiments to include more substantial patterns, such as for example, “checkerboard” stripes. Such patterns may provide better contrast for applications 199 to resolve in some applications. Additional examples of grid placement and image deconvolution may be found in U.S. Pat. No. 6,611,767; and U.S. Provisional Patent Application Ser. No. 60/578,816, titled “System, Method, and Computer Software Product for Genotyping and Genotype Data Visualization”, filed Jun. 10, 2004; both of which are hereby incorporated by reference herein in its entirety for all purposes.

For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly.

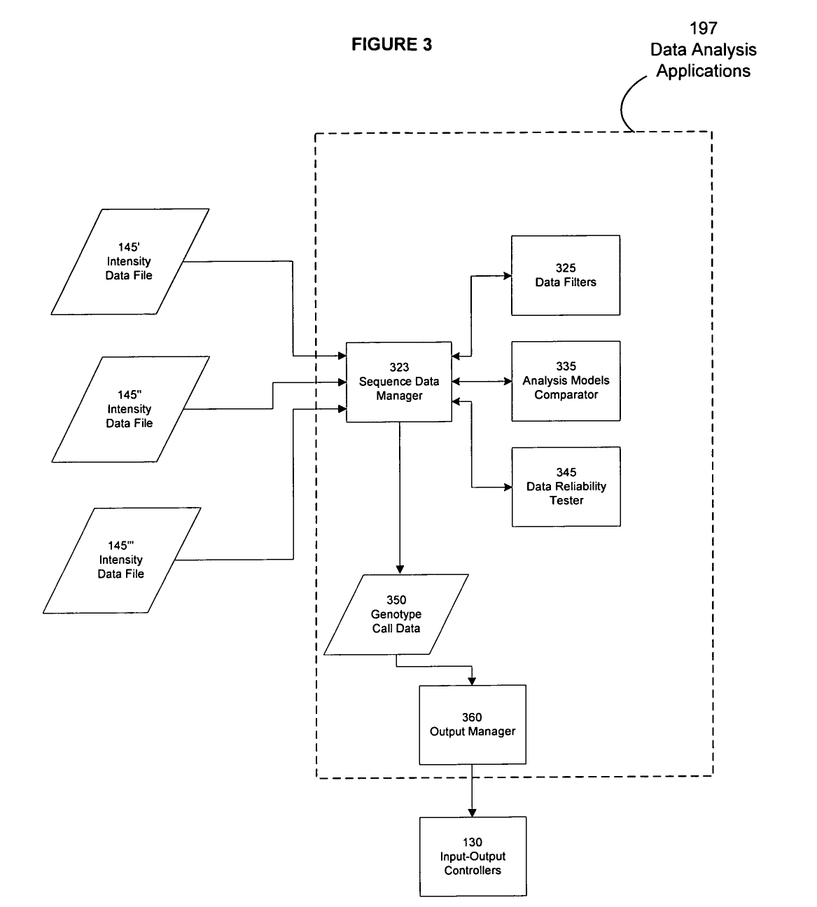

FIG. 3 further illustrates an example that may include intensity data file 145′, 145″, and 145′″. Each of data files 145 may contain emission intensity data for each probe feature disposed upon probe array 152. In the present example data file 145′ may correspond to a particular probe array type where an experimental sample has been tested. Additionally, data file 145″ and 145′″ may correspond to the same probe array type where different experimental samples have been used that may allow for the comparison between experimental samples. Those of ordinary skill in the related art will appreciate that each of files 145 may include one or more data files that may correspond to one or more experimental samples and/or arrays 152.

Also, files 145 may be further processed by some implementations of executables 199A. For example, the processing result may include what is referred to as a .chp file comprising values representative of degrees of hybridization, absolute and/or differential (over two or more experiments) expression, genotype values or determinations, detection of polymorphisms and mutations, and other similar analytical results. Further details regarding cel files, and .chp files, are provided in U.S. patent application Ser. No. 10/219,882 incorporated by reference above. In the present example, in which executables 199A include Affymetrix® Microarray Suite or GCOS, the .chp file is derived from analysis of the cel file combined in some cases with information derived from library files 143, such as for instance probe array design information that may identify each probe feature and location, as well as laboratory or other experiment data, or other types of information useful in the analysis and interpretation of data.

In some embodiments of executables 199A, user 175 may specify an implementation of probe array 152 such as an Affymetrix catalogue or custom chip type (e.g., Human Genome U133 plus 2.0 chip) either by selecting from a predetermined list presented by executables 199A, such as for instance in a graphical user interface, or by scanning a bar code, Radio Frequency Identification (RFID), magnetic strip, or other means of electronic identification related to a chip to read its type. Executables 199A may associate the chip type with various scanning parameters stored in data tables or library files, including the area of the chip that is to be scanned, the location of chrome borders or other features on the chip used for auto-focusing, the wavelength or intensity/power of excitation light to be used in reading the chip, and so on. As noted, applications 199 may apply some of this data in the generation of intermediate results.

For example, a bar code (or other machine-readable information such as may be stored on a magnetic strip, in memory devices of a radio transmitting module, or stored and read in accordance with any of a variety of other known techniques) may be affixed to the probe array, a cartridge, or other housing or substrate coupled to or otherwise associated with the array. The machine-readable information may automatically be read by a device (e.g., a 1-Dimensional, 2-Dimensional, or other type of bar code reader) incorporated within the scanner, an autoloader associated with the scanner, an autoloader movable between the scanner and other instruments, and so on. In any of these cases, executables 199A may associate the type of probe array 152, or other identifier, with various scanning parameters stored in data tables. The scanning parameters may include, for example, the area of the chip that is to be scanned, the starting place for a scan, the location of chrome borders on the chip used for auto-focusing, the speed of the scan, a number of scan repetitions, the wavelength or intensity of laser light to be used in reading the chip, and so on. In some embodiments, rather than storing this data in databases or data tables, some or all of it may be directly accessible from the machine-readable information coupled or associated with probe arrays 152. Other experimental or laboratory data may also include, for example, the name of the experimenter, the dates on which various experiments were conducted, the equipment used, the types of fluorescent dyes used as labels, protocols followed, and numerous other attributes of experiments.

As noted, executables 199A may apply some of this data in the generation of intermediate results. Other data, such as the name of the experimenter, may be processed by executables 199A or may simply be preserved and stored in files or other data structures. Any of these data may be provided, for example over a network such as network 280, to a laboratory information management server computer, configured to manage information from large numbers of experiments. A data analysis program may also generate various types of plots, graphs, tables, and other tabular and/or graphical representations of analytical data. As will be appreciated by those skilled in the relevant art, the preceding and following descriptions of files generated by executables 199A are exemplary only, and the data described, and other data, may be processed, combined, arranged, and/or presented in many other ways.

Data Analysis Applications 197: The processed image files produced by applications 199 often are further processed to extract additional data. In particular, data analysis applications 197 may be employed for specialized or supplemental identification and analysis of biologically interesting patterns or degrees of hybridization of probe sets. An example of a software application of this type is the Affymetrix® Data Mining Tool, described in U.S. patent application, Ser. No. 09/683,980, and Affymetrix® GeneChip® Data Analysis Software (hereafter referred to as GDAS), described in U.S. patent application Ser. No. 10/657,481, titled “System, Method, and Computer Software Product for Analysis and Display of Genotyping, Annotation, and Related Information”, filed Sep. 8, 2003; and U.S. patent application Ser. No. 10/986,963, titled “System, Method, and Computer Software Product for Generating Genotype Calls”, filed Nov. 12, 2004, each of which is hereby incorporated herein by reference in its entireties for all purposes. Embodiments of applications 197 include executable code being stored in system memory 120, illustrated in FIG. 1 as data analysis applications executables 197A.

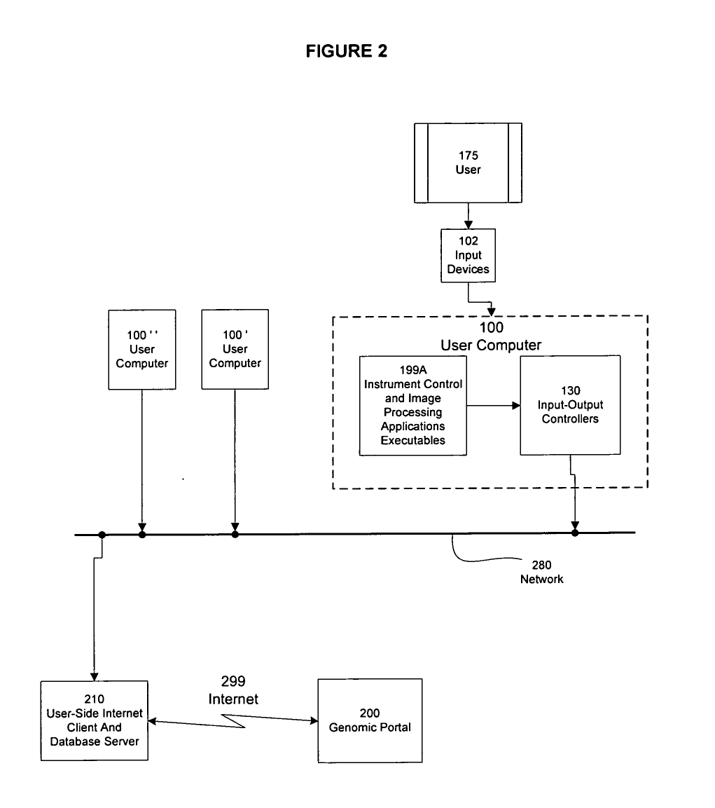

As will be appreciated by those skilled in the relevant art, it is not necessary that applications 197 be stored on and/or executed from computer 100; rather, some or all of applications 197 may be stored on and/or executed from an applications server or other computer platform to which computer 100 is connected in a network. For example, it may be particularly advantageous for applications involving the manipulation of large databases to be executed from a database server such as user-side internet client and database server 210 of FIG. 2. Alternatively, LIMS, DMT, and/or other applications may be executed from computer 100. But some or all of the databases upon which those applications operate may be stored for common access on server 210 (perhaps together with a database management program, such as the Oracle® 9i or 10g database management system from Oracle Corporation). Such networked arrangements may be implemented in accordance with known techniques using commercially available hardware and software, such as those available for implementing a local-area network or wide-area network. For example, a local network is represented in FIG. 2 as network 280 by the connection of user computer 100 to database server 210 (and to a user-side Internet client, which is illustrated in FIG. 2 as the same computer but need not be). The connections of network 280 could include a network cable, wireless network, or other means of networking known to those in the related art. Also, in the present example a wide area network may include internet 299 as is well known to those of ordinary skill in the related art. Similarly, scanner 150 (or multiple scanners) may be made available to a network of users over network 280 or internet 299 both for purposes of controlling scanner 150 and for receiving data input from it.

In some embodiments, executables 197A may communicate with one or more remote computers, serves, or other computing devices via user-side internet client 210 and internet 299. For example, user 175 may desire to acquire annotation information related to one or more target molecules, sequences, genes, chromosome, or other related type of biological information. Executables 197A may provide user 175 with one or more selectable fields, windows or other means for the input of query information, in a graphical user interface. In the present example, user 175 may clink on a name or identifier of a probe set or target molecule, where that identifier comprises a dynamic link to information provided by a remote server serviced by an internet portal such as genomic portal 200. Genomic portal 200 may return information in response to the query to executables 197A that may then in turn be displayed to user 175 in the same or other graphical user interface.

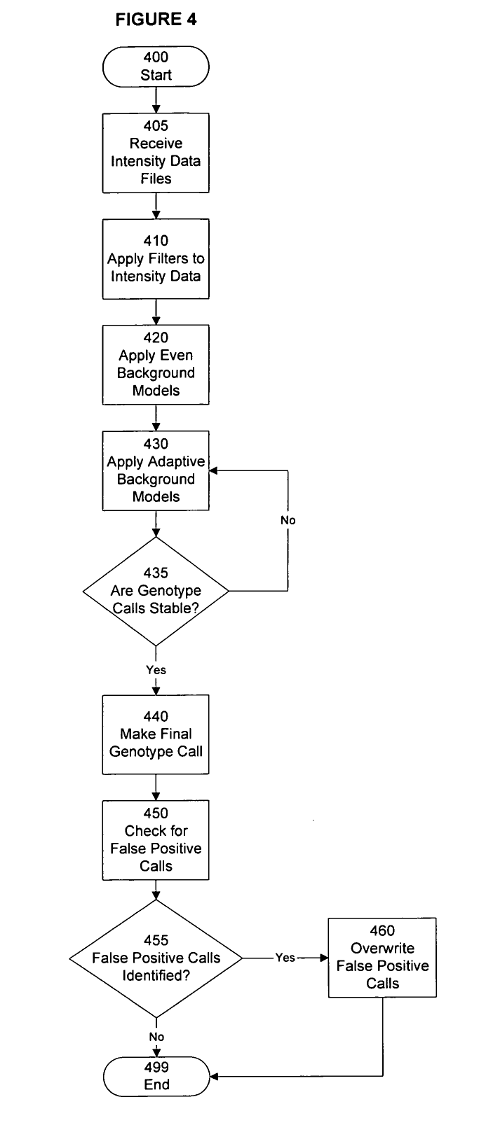

Sequence Data Manager 323: Some embodiments of data analysis applications executables 197A may include sequence data manager 323. In one embodiment sequence data manager 323 may manage the functions of analyzing the intensity values, illustrated in FIG. 3 as data file 145′, data file 145″, and data file 145′″. As illustrated in step 405 of FIG. 4, sequence data manager 323 receives each of data files 145 that represent the intensity data from a probe array experiment conducted on an individual sample. Data manager 323 may concurrently analyze a plurality of samples that could, for instance, include 200 or more samples. Also, some embodiments of sequence data manager 323 may process the data associated with each strand (strands may be referred to as the coding and non-coding strands; forward and reverse strands; or sense and anti-sense strands) independently. For example, manager 323 performs the analysis of each strand by applying one or more filters and models using a set of assumptions based on what may be referred to as an even background and uneven backgrounds. In the present example, manager 323 processes each strand independently and combines the independent results to produce a single genotype base call. Also in the present example, the independent processing of the strands accounts for differences in the background associated with each strand and therefore produces more reliable genotype calls and reduces the number of false positive calls.

In some embodiments genotyping algorithms may be applied to each of files 145 generated from particular embodiments of probe array 152 to identify the nucleic acid composition of a selected DNA sequence, single nucleotide polymorphisms (hereafter referred to as SNP's), or other features related to aspects of genomic sequence. For example, one type of algorithm could include the CustomSeq™ algorithm from Affymetrix, Inc. The CustomSeq™ algorithm may be used to determine nucleic acid composition for each sequence position of a selected DNA sequence. In the present example, the algorithm may use the intensity data values derived from probe sets disposed on probe arrays designed to interrogate specific genomic DNA or other type of sequences. The emission intensity data values may be contained within one or more data files that could for instance include *.cel file.

Data Filters 325: In some embodiments, manager 323 may implement one or more genotyping algorithms for the analysis of intensity data values, where at least one of the algorithms is performed in a number of steps. For example, manager 323 may initially employ data filters 325 to identify unreliable data or adjust what may be referred to as the variance associated with the intensity values, in particular values that may approach the limits of detection sometimes referred to as dynamic range of a detection instrument.

The term “variance” as used herein generally refers to a value that is a measure of the dispersion of data. For example, it will be appreciated by those skilled in the relevant art that, variance may be defined as the mean of the square of the differences between the samples and their mean and can be mathematically represented as:

$${σ}^{2} = \frac{\sum {( X - \overline{X} )}^{2}}{n - 1} \qquad \text{Equation-1}$$

where, X is equal to a particular value that could for instance be an intensity value for a probe feature.

\overline{X} is equal to the mean of all the values

n is equal to the total number of values.

As previously discussed, each implementation of probe array 152 may include a plurality of probes enabled to interrogate the nucleotide composition of particular nucleic acid sequence or SNP positions. In some embodiments, probes may be enabled to interrogate the sequence composition represented on each of the two complementary strands as is typically understood with respect to “Watson-Crick Base Pairing Rules”. For example, those of ordinary skill in the related art will appreciate that the strands may be referred to as the sense strand and the anti-sense strand and similarly may also be referred to as a coding and non-coding strand or forward and reverse strands of DNA. It will also be appreciated that the sense, coding, or forward strand of DNA generally refers to the strand that is read by the cellular machinery resulting in gene products namely protein from a single stranded m-RNA, while the anti-sense, non-coding, or reverse strand generally refers to the complementary strand. Those of ordinary skill in the related art will also appreciate that these are not strict definitions where there may also be some transcriptional products from the anti-sense, non-coding, or reverse strands, and therefore the terms should not be interpreted as limiting.

Some embodiments of data filters 325 may employ intensity values derived from one or more probe sets (set of probes which may be more than two in number and may contain any number of probes) associated with a particular sample to rule a sequence position as a no call or to make an adjustment to the variance value calculated for the values in a plurality of files 145. For example, data filters 325 may account for intensity values from two different probe sets that interrogate the same relative position in the genomic sequence, such as a probe set that interrogates a sequence position on the coding or sense strand, and a second probe set that interrogates the corresponding sequence position on the non-coding or the anti-sense strand.

Illustrated as step 410 of FIG. 4, data filters 325 may, for example, filter the intensity values in each embodiment of data file 145 to remove data that may otherwise negatively affect the quality of genotype call. Such data may be classified into categories of characteristics such as a no signal category, a weak signal category, a saturated signal category, or a high signal to noise ratio category. In the present example, data filters 325 may determine that the intensity value for a particular sequence position on a particular strand falls into one or more of the categories and rule the sequence position for that strand as a “no call”. Data filters 325 may then record the “no call” result in sample genotype call data 350 and/or employ the result in further analysis when data from each strand in combined.

The term “signal to noise ratio” as used herein generally refers to a ratio of intensity values associated with signal detected from the hybridized probes of probe array 152 to the intensity values associated with what is generally referred to as noise that may be regarded as artifacts. Source of noise may include fluorescent emissions generated from residual unbound sample, the non-specific binding of sample to probe features, what is referred to as “Dark Current” in instrument detectors or other electronic sources, or other processes generally known to those of ordinary skill. For example, data filters 325 may use a threshold value such as a pre-defined value or a user selected value. In the present example, a signal to noise ratio of 20 may be employed as a threshold value.

The “no signal” category may employ what may be referred to as a mean of the intensity values as a threshold value. For example, a mean intensity value may be defined as the mean value of the emission intensity values for all pixels within a probe feature. The threshold value may include a pre-defined value or a value computed from the data, such as a value that is within two standard deviations of zero. Alternatively the threshold value could be a value that the user selects. The term “standard deviation” as used herein generally refers to a value that is the square root of the variance. In the present example, the standard deviation value may be calculated from intensity data from each probe feature of the one or more probe sets that interrogates a particular sequence position from one or more samples. Alternatively the standard deviation value may be calculated from a subset of probe features such as a probe feature that interrogates a type of nucleic acid (i.e. A, C, G, or T), a probe set that interrogates the sequence of a particular strand (i.e. coding or non-coding strand), or from all probe sets of the probe array. If, data filters 325 determines that the mean intensity value for any probe feature of a probe set is below the threshold value then data filters 325 assigns the corresponding sequence position as a “no call”.

The “weak signal” category may employ what may be referred to as the highest mean intensity value. For example, the highest mean intensity value may be defined as the mean intensity value for a probe feature that is higher than all other mean intensity values of probe features in a probe set. The threshold value may include a pre-defined value or a value computed from the data, such as a value equal to a 20 fold decrease from the average highest mean intensities for all probe sets from the same strand (i.e. coding or non-coding strands). Alternatively, the threshold value may be a value that is selected by the user. In the present example, if data filters 325 determines that the highest mean intensity value for a probe set is below the threshold value then data filters 325 assigns the corresponding sequence position as a “no call”.

The “saturation” category may employ a threshold value that a plurality of probe features of a probe set fails in order for data filters 325 to assign the sequence position as a “no call”. For example, the threshold value could include a pre-defined value or a value computed from the data, such as a value that is two standard deviations below 43,000. The value of 43,000 is used in the present example as a representation of an intensity value that is at the limit of detection for a scanning system, but those of ordinary skill in the related art will appreciate that other values may be employed that are representative of the detection limits of particular instruments. As in the previous categories the user may also select the threshold value. The standard deviation value may be the same as that used for the no signal category, or may be different being derived from another set of emission intensity values.

A second criterion for the “saturation” category may also include a value of a maximum number of probe features that exceed the threshold value in order for a “no call” to be assigned to the sequence position. For example, if two or more probe features of the one or more probe sets associated with a single strand have mean intensity values greater than the threshold value then data filters 325 assigns the sequence position as a “no call”. Also if three or more features of the one or more probe sets associated with both strands are higher than the threshold value the data filters 325 assigns the sequence position as a “no call”.

Analysis Model Comparator 335: Illustrated in FIG. 4 as step 420, sequence data manager 323 may then forward the filtered emission intensity data to genotype call generator 335 to perform the next steps. The processes performed by comparator 335 may employ models developed to specify the presence or absence of specific nucleic acids in each sequence position of a selected DNA sequence. Different sets of models may be applied to the data based upon different assumptions. The assumptions may be based upon what may be referred to as an even background or uneven background that will be explained in more detail below.

In one embodiment, comparator 335 may calculate what may be referred to as a maximum likelihood function associated with each genotype state in order to determine the most likely genotype call. For example, the likelihood may be determined for the probe set intensity data associated with the sense and the anti-sense strands independently for a plurality of different states, each represented by a model, in order to determine the model that best fits the data. The likelihood and log-likelihood functions are the basis for deriving estimators for the data. Both these functions have a common maximum point. The maximum point known to those skilled in the relevant art as the Maximum Likelihood estimate (MLE) may be defined as the “most likely” value relative to the others. Therefore the state with the maximum likelihood may be the model that best fits the state. For example, null, A, C, G, and T may be the models assigned to the homozygous states and, null, AC, AT, AG, CT, CG, GT may be the models assigned to the heterozygous states. In the present example, one sequence state or model such as, for instance model A, may generally refer to what those of ordinary skill in the related art as describe a “consensus” or “wild type” sequence and all others may be referred to as what is described as a “mutant” sequence.

Comparator 335 may calculate the maximum likelihood for each of the models using intensity data from a plurality of probe sets for each sequence position associated with each strand from files 145. For example, each of the models comprises a set of assumptions that are true if the data fits the model. In the present example, a probe set may be comprised of four features or cells each representing a type of nucleic acid, where each of the cells is independent of each other. Each of the models assumes the pixel signal intensities for any given cell are independent, identically distributed, normal random variables. Further, each of the models may also assume that the sense and anti-sense sequences or strands are independent of each other, and the cells referred to as foreground cells in each of the models have a mean intensity value above some threshold value as determined previously by data filters 325. Similarly the cells referred to as the background cells in each of the models include mean intensity value below a threshold value. Additionally, for each of the models it may be assumed that both the foreground and the background cells are evenly distributed, in other words, all foreground cells have the same distribution (i.e. a Gaussian distribution), and all of the background cells have the same distribution (i.e. a Gaussian distribution).

Comparator 335 may perform calculations employing the emission intensities from each pixel for each cell of each probe set. For example, the calculations may include an observed mean μₓ, observed variance σₓ², estimated mean \hat{μ}ₓ, estimated variance σₓ², and number of observations nₓ that may, for instance, include the number of pixels in a cell. For both the observed and the estimated conditions, x includes the representation of the cells being considered. In the present example, each probe set may comprise 4 probe cells (i.e. probe features that represent four nucleic acid types A, G, C, and T) and therefore x=A, G, C, T. It will be appreciated by those skilled in the related art that a log-likelihood of the maximum likelihood function may help to link the data, unknown model parameters and assumptions and hence allows rigorous, statistical inferences. Therefore, it will be known to those skilled in the relevant art that, in order to minimize the estimation error, an explicit log-likelihood function for a probe set may be given by,

$$ll ( m ) = - \frac{1}{2} {\sum }_{x = [ A , G , C , T \}} {η}_{x} [ ln ( 2 {\hat{σ}}_{x}^{2} ) + \frac{{σ}_{x}^{2 } + {( {μ}_{x} - {\hat{μ}}_{x} )}^{2}}{{σ}_{x}^{2}} ] \qquad Equation \text{-} 2$$

Where, the sum is over all four cells for a given nucleic acid, and ρₓ is the number of pixels observed in probe feature x,

Comparator 335 may derive μₓ, and σₓ by solving the following equations:

$$\frac{δ ll ( m )}{δ {\hat{μ}}_{x}} = 0 , \frac{δ ll ( m )}{δ {\hat{σ}}_{x}} = 0 , \qquad Equation \text{-} 2.1$$

It will be appreciated by those of ordinary skill in the relevant art that the assumptions for an even background may be derived from what is referred to as the central limit theorem that generally allows making inferences about population means using the normal distribution no matter what the distribution of the population being sampled from. For example, each probe feature or cell of a probe set comprises a plurality of probes with identical sequence composition that may be relatively independent in their chance of binding a labeled target. Therefore as will be appreciated by those of ordinary skill in the related art, the overall emission intensity of the feature should be normally distributed (i.e. the probes have an equal chance of binding to the target molecules in the sample). Accordingly the central limit theorem may be applied to different models mentioned, for example, Null model, homozygous model, heterozygous model in order to obtain the corresponding equations.

Null Model: The maximum likelihood estimators for the null model where all the cells are assumed as background and evenly distributed may have a mean and variance of

$$\hat{ μ} ≡ {\hat{μ}}_{A} = {\hat{μ}}_{C} = {\hat{μ}}_{G} = {\hat{μ}}_{T} = \frac{{\sum }_{x = \{ A , C , G , T \}} {η}_{x} {μ}_{x}}{{\sum }_{x = \{ A , C , G , T \}} {η}_{x}} \\ {\hat{σ}}_{x}^{2} ≡ {\hat{σ}}_{xA}^{2} = {\hat{σ}}_{xC}^{2} = {\hat{σ}}_{xG}^{2} = {\hat{σ}}_{xT}^{2} \\ = \frac{{\sum }_{x = \{ A , C , G , T \}} {η}_{x} [ {σ}_{x}^{2} + {μ}_{x}^{2} ]}{{\sum }_{x = \{ A , C , G , T \}} {η}_{x}} - {\hat{μ}}^{2} \qquad Equation \text{-} 3$$

Homozygote Model: The homozygote models may be similar to the no call model, but with slightly different assumptions, where 1 cell may be considered foreground and the other three cells are considered background. For example, the maximum likelihood estimators for the homozygote A model where the A cell is foreground and the C, T, and G cells and considered background, and may include a mean and variance of
\hat{μ}_{A}=μ_{A}, \hat{σ}_{A}²=σ_{A}² Equation-4:

Similarly the mean and variance of probe set cells C, G and T may be considered as background and evenly distributed, where the mean and variance of probe set cells C, G, and T may be given by,

$${\hat{ μ}}_{C} = {\hat{μ}}_{G} = {\hat{μ}}_{T} = \frac{{\sum }_{x = \{ C , G , T \}} {η}_{x} {μ}_{x}}{{\sum }_{x = \{ C , G , T \}} {η}_{x}} \\ {\hat{σ}}_{xC}^{2} = {\hat{σ}}_{xG}^{2} = {\hat{σ}}_{xT}^{2} = \frac{{\sum }_{x = \{ C , G , T \}} {η}_{x} [ {σ}_{x}^{2} + {( {\hat{μ}}_{x} - {μ}_{x} )}^{2} ]}{{\sum }_{x = \{ C , G , T \}} {η}_{x}} \qquad Equation \text{-} 4.1$$

The same likelihood estimation process may apply to the other homozygous models for each of the remaining nucleic acids, for example, the C, G, or T models where, for instance, probe set cell 3 may be associated with the C nucleic acid and considered foreground while the other three probe set cells, for example, A, G and T are assumed as background with an even distribution.

In the present example, if the estimated mean for the model is less than the estimated mean of the background, then the likelihood is set to the “no call” model.

Heterozygote Model: The heterozygote model, assumes that two of the cells are foreground and the remaining two are background. For example, with respect to an A/C heterozygote model, the cells associated with A and C are foreground and evenly distributed, and the cells for T and G are background and evenly distributed. Therefore maximum likelihood estimators for the A/C model may be given as,

For Probe Set Cells A and C:

$${\hat{ μ}}_{A} = {\hat{μ}}_{C} = \frac{{η}_{A} {μ}_{A} + {η}_{C} {μ}_{C}}{{η}_{A} + {η}_{C}} \\ {\hat{ σ}}_{A}^{2} = {\hat{σ}}_{C}^{2} \\ = \frac{{η}_{A} [ {σ}_{A}^{2} + {( {\hat{μ}}_{A} - {μ}_{A} )}^{2} ] + {η}_{C} [ {σ}_{C}^{2} + {( {\hat{μ}}_{C} - {μ}_{C} )}^{2} ]}{{η}_{A} + {η}_{C}} \qquad Equation \text{-} 5$$

For Probe Set Cells G and T:

$${\hat{ μ}}_{G} = {\hat{μ}}_{T} = \frac{{η}_{G} {μ}_{G} + {η}_{T} {μ}_{T}}{{η}_{G} + {η}_{T}} \\ {\hat{σ}}_{G}^{2} = {\hat{σ}}_{T}^{2} = \frac{{η}_{G} [ {σ}_{G}^{2} + {( {\hat{μ}}_{G} - {μ}_{G} )}^{2} ] + {η}_{T} [ {σ}_{T}^{2} + {( {\hat{μ}}_{T} - {μ}_{T} )}^{2} ]}{{η}_{G} + {η}_{T}} \qquad Equation \text{-} 5.1$$

The log-likelihood functions may have a single mode or maximum point and no local optima and therefore maximizing the likelihood functions can get the best fit model with optimal outcome. Hence maximum likelihood estimators may be obtained for all parameters in different states.

Those of ordinary skill in the related art will appreciate that the models for the other heterozygote combinations are similar to those illustrated in Equations 5, and 5.1.

Continuing the example from above, comparator 335 assigns an initial base call for each sequence position associated with each strand.

Step 430 of FIG. 4 illustrates the application of uneven background models to the intensity data in each of files 145. It may be desirable in many applications to apply what may be referred to as “adaptive background” methods to the data to account for differences that cause unevenness in the background between bases and/or samples. Possible sources of such background differences could come from what is generally referred to as cross hybridization, variation between each implementation of probe array 152, and other sources. For example, comparator 335 may apply the intensity data in each of files 145 for each probe set associated with each strand to a plurality of uneven background models in a manner similar to that as described above with respect to the even background models. In the present example, the mean and standard deviation values are calculated based upon a set of uneven background assumptions that may be employed in the calculation of log-likelihood values.

Homozygote Model: Similar to the even background models a model that represents the homozygous state may be employed, where for example for a model for homozygous A call may be represented as:
\hat{μ}_{C}=\hat{μ}_{bg}, \hat{σ}_{C}=\hat{σ}_{bg}²
\hat{μ}_{G}=β(G/C)\hat{μ}_{bg}, σ_{G}²=α(G/C)\hat{σ}_{bg}²
\hat{μ}_{T}=β(T/C)\hat{μ}_{bg}, σ_{T}²=α(T/C)\hat{σ}_{bg}² Equation-6

Where α and β are normalization coefficients between the background features, and \hat{μ}_{bg}, σ_{bg}² are parameters for the common even background. Using an assumption that α s and β s are constant, the \hat{μ}_{bg}, \hat{σ}_{bg}² parameters can be estimated as:

$${\hat{μ}}_{bg} = \frac{\begin{matrix}{η}_{C} {μ}_{C} α ( G / C ) α ( T / C ) + \\ {η}_{G} {μ}_{G} β ( G / C ) α ( T / C ) + {η}_{T} {μ}_{T} β ( T / C ) α ( G / C )\end{matrix}}{\begin{matrix}{η}_{C} α ( G / C ) α ( T / C ) + \\ {η}_{G} {β}^{2} ( G / C ) α ( T / C ) + {η}_{T} {β}^{2} ( T / C ) α ( G / C )\end{matrix}} \\ {\hat{σ}}_{bg}^{2} = \frac{{η}_{C} {ω}_{C} + {η}_{G} {ω}_{G} + {η}_{T} {ω}_{T}}{α ( G / C ) α ( T / C ) ( {η}_{C} + {η}_{C} + {η}_{T} )} \qquad Equation \text{-} 6.1$$

Where:
ω_{C}=α(G/C)α(T/C)[σ_{C}²+(μ_{C}−\hat{μ}_{C})²]
ω_{G}=α(T/C)[σ_{G}²+(μ_{G}−β(G/C)\hat{μ}_{G})²]
ω_{T}=α(G/C)[σ_{T}²+(μ_{T}−β(T/C)\hat{μ}_{T})²] Equation-6.2

The α and β normalization coefficients may be estimated as;

$$α ( G / C ) = \frac{\sum [ {η}_{G} {μ}_{G} ] \sum {η}_{C}}{\sum [ {η}_{C} {μ}_{C} ] \sum {η}_{G}} \\ β ( G / C ) = \frac{\sum [ {η}_{G} {μ}_{G} ] \sum {η}_{C}}{\sum [ {η}_{C} {μ}_{C} ] \sum {η}_{G}} \\ α ( T / C ) = \frac{\sum [ {η}_{T} {μ}_{T} ] \sum {η}_{C}}{\sum [ {η}_{C} {μ}_{C} ] \sum {η}_{T}} \\ β ( T / C ) = \frac{\sum [ {η}_{T} {μ}_{T} ] \sum {η}_{C}}{\sum [ {η}_{C} {μ}_{C} ] \sum {η}_{T}} \qquad Equation \text{-} 6.3$$

Where comparator 335 may take the summation as per the equations illustrated in equations 6.3 using the following rules:

1. Sum the values over absolute calls across all samples

2. If the values of less than 2 Samples have been summed, sum over marginal calls across all samples

3. If the values of less than 2 samples have been summed, sum over all “no calls”

Heterozygote Model: The heterozygote model for the uneven background is similar to the homozygote except that, in a manner similar to the even background models, only 2 background models are averaged rather than 3. Those of ordinary skill in the related art will appreciate the differences between the homozygous and heterozygous models. For example, the homozygous model for the A call must account for the C, G, and T, backgrounds where a heterozygous model for an A/C call has to account for the G, and T backgrounds.

In some embodiments, comparator 335 may iteratively repeat the adaptive background method, as illustrated in decision element 435, until the base or genotype calls are stable and do not change. For example, comparator 335 may perform a pre-set number of iterations, a user selected number of iterations, or a dynamic number of iterations that varies based upon the quality of data in files 145 where comparator 335 may determine a point where there is substantially no change in genotype calls across the samples from the last iteration.

In some embodiments, comparator 335 produces an intermediate genotype call for each probe set associated with the forward and reverse strands associated with a sequence position. For example, comparator 335 may compute what may be referred to as a reliability score for the sequence position interrogated by the probe sets for each strand, and make a base call using the reliability score. In the present example, comparator 335 may compute the reliability score for each strand as:
RS(M)=ll(M)−max{ll(m),m ε S,m ≠ M} Equation 7

Where M is the best fitting model for the strand that may be denoted as RS_{f} for the forward strand and RSᵣ for the reverse strand, and:
S={Null, A, G, C, T, AC, AT, AG, CT, CG, GT}

Additionally, comparator 335 may categorize the each strand base call into one of two levels or a “no call” using the following parameters:
Absolute Call: RS>τ₁
Marginal Call: RS>τ₂ Equation 7.1
No Call: all others Equation 7.1

In the present example τ₁ and τ₂ are threshold parameter values that may be pre-defined using, for instance, information determined mathematically or experimentally or in some embodiments selected by user 175.

Comparator 335 may then make a final base call for each sequence position represented in data files 145, where the processed values from both stands are combined to produce a single base or genotype call. For example, comparator 335 may apply the following rules to the intermediate calls from the uneven models for both strands:

1. If both forward and reverse strands are filtered out by data filters 325, comparator 335 assigns a “no call” and quality score is zero.

2. If one strand is filtered out by data filters 325, comparator 335 uses the other strand for both base call and quality score.

3. If both strands have the same call, comparator 335 uses the common call as base call and quality score is the sum of the two.

4. If the two calls are different heterozygote calls, comparator 335 assigns a “no call” and zero quality score.

5. If one call is homozygote, the other is heterozygote and the homozygote allele is one of the two in the heterozygote call, comparator 335 assigns the homozygote call and its quality score to that base.

6. If the quality score is less than a threshold or cutoff value α, then comparator 335 assigns a “no call”.

Data Reliability Tester 345: In some embodiments, sequence data manager 323, may forward the final base calls generated by comparator 335 to data reliability tester 345 in order to test the reliability of the genotype calls, and in particular test for what may be referred to as “false positive calls” illustrated as step 450. Some embodiments of data reliability tester 345 may employ what may be referred to as a “wild type sequence profile and trace” in order to determine the reliability of each base call. For example, data reliability tester 345 may use what may be referred to as a reference or consensus sequence that is representative of the expected sequence composition of the sequence interrogated by probe array 152. Data reliability tester 345 employs the reference sequence for comparison with each of the final base or genotype calls to determine a measure of confidence that the call is either accurate or a false positive.

For example, a wild type sequence profile may be defined by taking what those of ordinary skill in the related art refer to as a “Centered Exponentially Weighted Moving Average” of the intensity values for the probe features with the reference bases in order to “smooth” the intensity values of the probe feature that interrogates the wild-type base, where data reliability tester 345 takes the average using a “window” that is moved along the sequence. The term “window” as used herein generally refers to a number of sequence positions that define an area where the number of sequence positions could refer to a total number of sequence positions, a number of sequence positions on either side of a central base position, or other number. Data reliability tester 345 also “smoothes” the intensity values of the probe feature that interrogates what may be referred to as a potential “mutant” base. In the present example, a potential mutant base may be defined as the probe feature with the highest intensity values among the three probe features that do not represent the wild-type base. The equations for smoothing the wild-type and potential mutant signals may be given as:

$${I}_{wt}^{s} ( x ) = {\sum }_{i = - k}^{k} {α ( 1 - α )}^{\vert i \vert} {I}_{wt}^{0} ( x + i ) , \\ {I}_{mt}^{s} ( x ) = {\sum }_{i = - k}^{k} {α ( 1 - α )}^{\vert i \vert} {I}_{mt}^{0} ( x + i ) \qquad Equation \text{-} 8$$

Where x represents the index for a given base; k represents the size of the window as a value associated with the number of bases; α is a constant that performs as a smoothing weighting factor that may in some embodiments include a value of 0.5 (a value of 1 may be associated with no smoothing); “s” represents smoothing, “o” represents observed, “wt” represents wild-type, “mt” represents mutant; “I” represents the intensity value. Also:
Iₘₜ^{o}(y)=max{I_{b}^{o}(y): b ε Ω & b ≠ wt} Equation 8.1

Where b represents the bases that are not the wild-type base.

Continuing the example from above, a wild-type sequence profile may calculated that may be defined as the log ratio of the smoothed wild-type value and the smoothed potential mutant value, where the wild-type sequence profile F(X) may be defined as:

$$F ( x ) = log ( \frac{{I}_{wt}^{s} ( x )}{{I}_{mt}^{s} ( x )} ) \qquad \text{Equation - 8.2}$$

and further, the wild-type trace Tr(X) may be defined as:

$$\begin{matrix}Tr ( x ) = \frac{{F}_{-}^{′} ( x ) + {F}_{+}^{′} ( x )}{2} & \text{Equation - 8.3} \\ where \text{:} & \\ {F}_{-}^{′} ( x ) = \frac{F ( x ) - F ( x - Δ x )}{Δ x} , & \text{Equation - 8.4} \\ {F}_{+}^{′} ( x ) = \frac{F ( x ) - F ( x + Δ x )}{Δ x} & \end{matrix}$$

Where Δx represents the step size for the derivatives (in some embodiments may include a default value of 2).

Still continuing the example from above, data reliability tester 345 identifies false positives by application of the wild-type sequence profile and wild-type trace to the following rules:
F(x)>δ or Tr(x)>ε, Equation-8.5

Where ^{δ} and ε are constant values that may be pre-defined or user selected.

Data reliability tester 345 may determine that a base call is a false positive if either of the conditions illustrated in Equation 8.5 are true. As illustrated in decision element 455, if data reliability tester 345 does not find any false positive calls for any of the base calls, then the method is complete. Alternatively, any base call that data reliability tester 345 determines to be a false positive call is overwritten and assigned as a “no call”.

Sequence data manager 323 may then assemble the results from data reliability tester 345 into one or more genotype call data files 350. Data 350 may comprise the results that correspond to all samples, or alternatively there may be a separate data file 350 that corresponds to each sample. For example, the genotype call results from intensity data files 145′, 145″, and 145′″ may be combined into one sample genotype data file 350. In the present example, that could be a separate sample genotype data file 350 for each intensity data files 145.

Output Manager 360: Output manager 360 may then receive the one or more data files 350 from manager 323.

In some embodiments the output manager 360 may arrange the genotype calls from each sample and pass them to the input-output controllers 130. Controllers 130 then correspond with the display devices 180 to present the user with the genotype results in a graphical user interface, such as GUI 182.

Many visualization tools are available that present the user with the results of the analysis. However a user friendly visualization tool is required that aids the user to easily understand the results, the overall quality of the results and provides the user a level of flexibility to decide the parameters, for example, the cut off value in order to obtain the desired genotype results. This tool may further aid in the linkage study or the association when applied to the whole genome and may give an in depth coverage of the whole genome being studied.

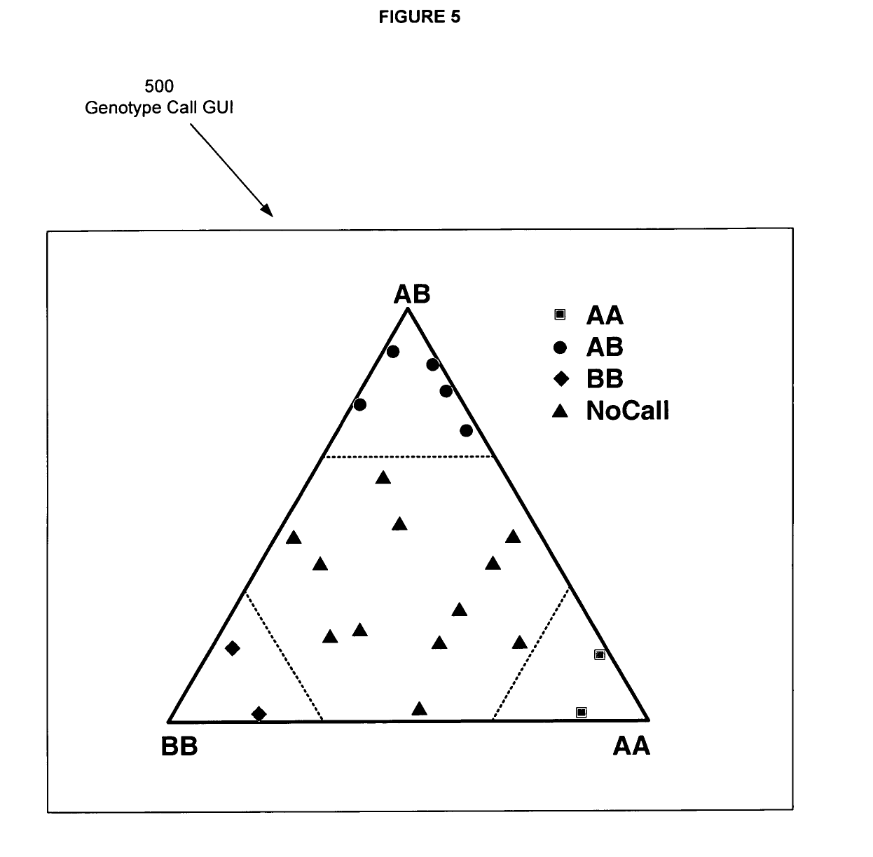

One possible example of a presentation of genotype call data is presented in FIG. 5 as genotype call GUI 500. For example, GUI 500 may provide user 175 with a visual representation of the confidence level of one or more of the genotype or base calls in genotype data file 350. The representation may comprise a triangular shape with genotype calls represented at each of the vertices, as well as a visual representation of a threshold for bases assigned as a “no call”. In the present example, manager 360 may calculate confidence scores for each of the genotype base calls for display, where the confidence score for a base call is represented by the relative distance from its associated vertex such as for instance, high confidence scores are represented closest to the vertex and low confidence scores are represented farther away from the vertex.

Further examples of the representation of GUI 500 and the calculation of confidence scores are described in U.S. Provisional Patent Application Ser. No. 60/578,816, titled “System, Method, and Computer Software Product for Genotyping and Genotype Data Visualization”, filed Jun. 10, 2004. Also, other graphical representations of genotype call data are described in U.S. patent application Ser. No. 10/986,963, titled “System, Method, and Computer Software Product for Generating Genotype Calls”, filed Nov. 12, 2004, each of which is incorporated by reference above.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments. For example, some or all of the functions described as being carried out by output manager 360 could be carried out by sequence data manager 323, or these functions could otherwise be distributed among other functional elements. Also, the functions of several elements may, in alternative embodiments, be carried out by fewer, or a single, element. For example, the functions of output manager 360 and sequence data manager 323 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the functions performed by the two servers could be performed by a single server or other computing platform, distributed over more than two computer platforms, or other otherwise distributed in accordance with various known computing techniques.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

1) A method for calling the genotype of a biological sequence, comprising;
a) receiving one or more sets of intensity data each comprising an intensity value for each of a plurality of probe features, wherein each probe feature is associated with a probe set disposed on a probe array;
b) independently applying one or more filters to the intensity values of a probe set associated with a forward strand and the intensity values of a probe set associated with a reverse strand, wherein the probe sets for the forward and reverse strands interrogate a same sequence position;
c) independently applying one or more models to the filtered intensity values for each of the forward strand and the reverse strand, wherein the models produce a genotype call for the forward strand and a genotype call for the reverse strand;
d) combining the genotype call for the forward strand and the genotype call for the reverse strand to generate a final genotype call for the same sequence position; and
e) testing the reliability of the final genotype call.

2) The method of claim 1, further comprising:
f) repeating steps b-e for each probe set associated with each sequence position of the biological sequence.

3) The method of claim 2, further comprising:
g) providing a representation of one or more of the final genotype calls to a user.

4) The method of claim 1, wherein:
each of the one or more sets of intensity data comprise an intensity data file associated with a sample.

5) The method of claim 1, wherein:
each of the one or more filters identify unreliable data associated with a category selected from the group consisting of a signal to noise ratio category, a no signal category, a weak signal category, and a saturation category.

6) The method of claim 1, wherein:
the one or more models comprise an even background model and an uneven background model.

7) The method of claim 6, wherein:
the uneven background model is applied iteratively.

8) The method of claim 1, wherein:
the genotype call for the forward strand and the genotype call for the reverse strand are selected from the group consisting of a no call, a homozygous call, and a heterozygous call.

9) The method of claim 8, wherein:
the final genotype call comprises the genotype call of the forward strand or the genotype call of the reverse strand that is not a no call if the other genotype call is a no call.

10) The method of claim 8, wherein:
the final genotype call comprises the genotype call for the forward strand and the genotype call for the reverse strand if both strands comprise a same call.

11) The method of claim 8, wherein:
the final genotype call comprises a no call if the genotype calls for the forward and reverse strands are different heterozygote calls.

12) The method of claim 8, wherein:
the final genotype call comprises a homozygote call if the forward and reverse genotype calls include a homozygote call and a heterozygote call and an allele represented by the homozygote call is one of two alleles represented by the heterozygote call.

13) The method of claim 1, wherein:
the reliability of the final genotype call comprises testing for a false positive.

14) The method of claim 13, wherein:
the final genotype call is a no call if the false positive is true.

15) A system for calling the genotype of a biological sequence, comprising;
a) a data manager that receives one or more sets of intensity data each comprising an intensity value for each of a plurality of probe features, wherein each probe feature is associated with a probe set disposed on a probe array;
b) one or more data filters that independently apply one or more filters to the intensity values of a probe set associated with a forward strand and the intensity values of a probe set associated with a reverse strand, wherein the probe sets for the forward and reverse strands interrogate a same sequence position;
c) a comparator that independently applies one or more models to the filtered intensity values for each of the forward strand and the reverse strand, wherein the models produce a genotype call for the forward strand and a genotype call for the reverse strand, and further wherein the comparator combines the genotype call for the forward strand and the genotype call for the reverse strand to generate a final genotype call for the same sequence position; and
e) a reliability tester that tests the reliability of the final genotype call.

16) The system of claim 15, further comprising:
g) an output manager that provides a representation of one or more of the final genotype calls to a user.

17) The system of claim 15, wherein:
each of the one or more sets of intensity data comprise an intensity data file associated with a sample.

18) The system of claim 15, wherein:
each of the one or more filters identify unreliable data associated with a category selected from the group consisting of a signal to noise ratio category, a no signal category, a weak signal category, and a saturation category.

19) The system of claim 15, wherein:
the one or more models comprise an even background model and an uneven background model.

20) The system of claim 19, wherein:
the uneven background model is applied iteratively.

21) The system of claim 15, wherein:
the genotype call for the forward strand and the genotype call for the reverse strand are selected from the group consisting of a no call, a homozygous call, and a heterozygous call.

22) The system of claim 21, wherein:
the final genotype call comprises the genotype call of the forward strand or the genotype call of the reverse strand that is not a no call if the other genotype call is a no call.

23) The system of claim 21, wherein:
the final genotype call comprises the genotype call for the forward strand and the genotype call for the reverse strand if both strands comprise a same call.

24) The system of claim 21, wherein:
the final genotype call comprises a no call if the genotype calls for the forward and reverse strands are different heterozygote calls.

25) The system of claim 21, wherein:
the final genotype call comprises a homozygote call if the forward and reverse genotype calls include a homozygote call and a heterozygote call and an allele represented by the homozygote call is one of two alleles represented by the heterozygote call.

26) The system of claim 15, wherein:
testing the reliability of the final genotype call comprises testing for a false positive.

27) The system of claim 26, wherein:
the final genotype call is a no call if the false positive is true.

28) A method for calling the genotype of a biological sequence, comprising;
a) receiving one or more sets of intensity data each comprising an intensity value for each of a plurality of probe features, wherein each probe feature is associated with a probe set disposed on a probe array;
b) applying one or more models to each of the intensity values for the probe set associated with each of a forward strand and a reverse strand, wherein the models produce a genotype call for the forward strand and a genotype call for the reverse strand; and
c) combining the genotype call for the forward strand and the genotype call for the reverse strand to generate a final genotype call for the same sequence position.

29) The method of claim 28, further comprising:
d) repeating steps b-c for each probe set associated with each sequence position of the biological sequence.

30) The method of claim 29, further comprising:
e) providing a representation of one or more of the final genotype calls to a user.

31) The method of claim 28, wherein:
each of the one or more sets of intensity data comprise an intensity data file associated with a sample.

32) The method of claim 28, wherein:
the one or more models comprise an even background model and an uneven background model.

33) The method of claim 28, wherein:
the genotype call for the forward strand and the genotype call for the reverse strand are selected from the group consisting of a no call, a homozygous call, and a heterozygous call.

35) A system for calling the genotype of a biological sequence, comprising:
a computer comprising system memory having executable code stored thereon, wherein the executable code performs a method, comprising;
a) receiving a one or more sets of intensity data each comprising an intensity value for each of a plurality of probe features, wherein each probe feature is associated with a probe set disposed on a probe array;
b) independently applying one or more filters to the intensity values of a probe set associated with a forward strand and the intensity values of a probe set associated with a reverse strand, wherein the probe sets for the forward and reverse strands interrogate a same sequence position;
c) independently applying one or more models to the filtered intensity values for each of the forward strand and the reverse strand, wherein the models produce a genotype call for the forward strand and a genotype call for the reverse strand;
d) combining the genotype call for the forward strand and the genotype call for the reverse strand to generate a final genotype call for the same sequence position; and
e) testing the reliability of the final genotype call.
